(19) United States
(12) Patent Application Publication
(10) Pub. No.: US 2024/0164951 A1
(43) Pub. Date: May 23, 2024

(54) ADHESIVE TAPE

(71) Applicant: Berry Global, Inc., Evansville, IN (US)

(72) Inventors: Svetlana CONTRADA, Evansville, IN (US); Jill MCROBBIE, Evansville, IN (US); Paul CONNOLLY, Evansville, IN (US)

(21) Appl. No.: 18/514,092

(22) Filed: Nov. 20, 2023

Related U.S. Application Data
(60) Provisional application No. 63/426,806, filed on Nov. 21, 2022.

(51) Int. Cl. A61F 13/02

(52) U.S. Cl. CPC A61F 13/0269; A61F 13/0253; A61F 13/0259; A61F 13/0276

(57) ABSTRACT

The present disclosure provides improved adhesive tape compositions for application with medical devices, for instance wearable medical devices. The adhesive tape composition comprises i) a fabric and ii) an adhesive.

### CROSS-REFERENCE TO RELATED APPLICATIONS

This application claims the benefit under 35 USC § 119(e) of U.S. Provisional Application Ser. No. 63/426,806, filed on Nov. 21, 2022, the entire disclosure of which is incorporated herein by reference.

### BACKGROUND

Adhesive tape is utilized for many different purposes. One important usage of adhesive tape is for application to wearable devices, including medical devices. For instance, adhesive tape can be used to attach sensors of glucose monitoring medical devices to human skin in order to monitor a subject for the treatment of diabetes.

Currently available adhesive tapes for medical devices have limited duration when used with an attached medical device. For example, current adhesive tapes can have a fixed thickness of adhesive material, which can impede the ability of the tape to stick to the user's skin. As a result, medical devices must be undesirable re-applied to the skin multiple times and can impact the effectiveness of the monitoring by the devices. Further, adhesive tapes should be able to support the weight of a medical device and ensure accurate sensor readings on the user.

Accordingly, the present disclosure provides improved adhesive tape compositions for application with medical devices, for instance wearable medical devices. As described herein, the adhesive tape compositions can advantageously provide prolonged duration of use with wearable devices. Furthermore, the adhesive tape compositions are breathable and provide bi-directional stretch properties, which allows for secure attachment to the skin of a user and at the same time follows the movements of the user to provide longer periods of use for medical devices.

### BRIEF DESCRIPTIONS OF THE DRAWINGS

The detailed description particularly refers to the accompanying figures in which:

FIG. 1 is a cross section of an exemplary adhesive tape compositions of the present disclosure.

FIG. 2 is a scanning electron microscope (SEM) image of a cross section of an exemplary adhesive tape composition of the present disclosure.

FIG. 3 is the SEM image of FIG. 2 with additional information regarding the various elements of the exemplary adhesive tape compositions of the present disclosure.

FIG. 4 is an SEM image of a cross section of prior art composition 3M 9907T, demonstrating uniform thickness of fabric and adhesive.

FIG. 5 is an SEM image of a cross section of prior art composition 3M 9907W, demonstrating uniform thickness of fabric and adhesive.

FIG. 6 shows variable thickness of adhesive in view of the embossed pattern on Sample 5 (i.e., 3619A).

FIG. 7 shows uniform adhesive thickness for Sample 3 (i.e., Avery, MED 5740), which does not have an embossed pattern.

FIG. 8 shows uniform adhesive thickness for Sample 4 (i.e., Dermamed, DM2275), which does not have an embossed pattern.

FIG. 9 shows a cross section of the sample is cut with a razor along the dashed line, making sure to cut through the middle point of the embossed area.

FIG. 10 is an original sample image obtained to make measurements as described in Example 3.

FIG. 11 shows a drawn Line 1 between the two highest points of fabric surface on both sides of the emboss area to make measurements as described in Example 3.

FIG. 12 shows identification of the middle point of the emboss area to make measurements as described in Example 3.

### DETAILED DESCRIPTION

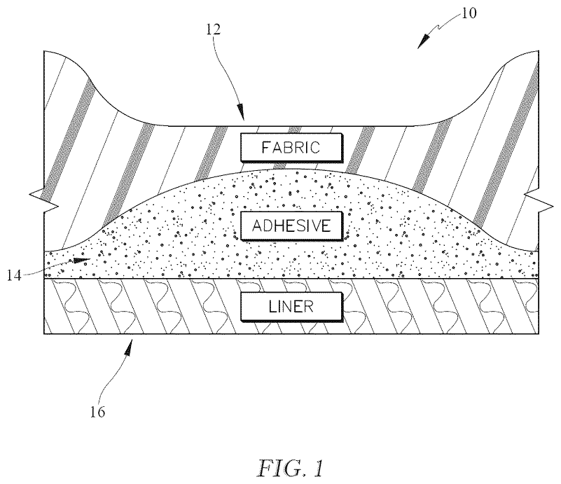

In an illustrative aspect, an adhesive tape composition 10 is provided. The adhesive tape composition comprises i) a fabric 12 and ii) an adhesive 14 as shown in FIG. 1. Further, the adhesive tape composition can comprise i) a fabric 12, ii) an adhesive 14, and iii) a removable liner 16 as shown in FIG. 1. In an embodiment, the fabric is a non-woven fabric. In an embodiment, the fabric is a breathable fabric. In an embodiment, the fabric comprises polyurethane. In an embodiment, the fabric comprises polyurethane resin. In an embodiment, the fabric comprises polyurethane resin compounded with one or more additives. In an embodiment, the additive is selected from the group consisting of a colorant, a dye, a pigment, an ultraviolet (UV) blocker, a UV absorber, or a functional filler, a polymer, and any combination thereof.

In an embodiment, a blend of polyurethane resin with polyethylene or other polypropylene resins can be utilized. Polyurethane is generally known in the art as providing desirable properties such as flexibility and breathability. However, other polymer composite materials (e.g., those based on polyester or polyolefins) can achieve breathability by processing or by incorporating functional additives. For instance, inorganic additives can be utilized with the embodiments of the present disclosure as a backing fabric or as a film of the adhesive tape.

In an aspect, fabrics comprising polyurethane can provide high moisture vapor transmission rate (MVTR) values, which allows for breathability of the tape and desirably permits for longer wear time on a user's skin and prevention of skin irritation. In an embodiment, the fabric is configured for bi-directional stretching.

Various fabrics are contemplated for the adhesive tape compositions of the present disclosure. For instance, a fabric can be obtained from Mativ Holdings, Inc. (Alpharetta, Georgia, U.S.A.).

The fabric can have various properties of certain ranges, including any of the following:

| Property | Range | |
|---|---|---|
| Basis Weight | 30-120 | gsm |
| | 40-110 | gsm |
| | 50-100 | gsm |
| | 60-90 | gsm |
| | 70-80 | gsm |
| Thickness | 100-1000 | microns |
| | 200-800 | microns |
| | 200-500 | microns |
| | 300-400 | microns |

| | |
|---|---|
| Young Modulus, MD | 0-10N/5 cm |
| | 0-5N/5 cm |
| | 2-5N/5 cm |
| | 5-10N/5 cm |
| Tensile Strength, MD | 10-150N/5 cm |
| | 20-100N/5 cm |
| | 25-90N/5 cm |
| | 30-80N/5 cm |
| | 40-50N/5 cm |
| Tensile Strength, TD | 5-150N/5 cm |
| | 10-125N/5 cm |
| | 15-100N/5 cm |
| | 20-80N/5 cm |
| | 25-75N/5 cm |
| | 30-70N/5 cm |
| | 40-60N/5 cm |
| Elongation at Break, MD | 50-1000% |
| | 100-1000% |
| | 200-1000% |
| | 300-1000% |
| | 400-1000% |
| | 500-1000% |
| Elongation at Break, TD | 50-1000% |
| | 100-1000% |
| | 200-1000% |
| | 300-1000% |
| | 400-1000% |
| | 500-1000% |

In an embodiment, the fabric comprises a front side and a back side, wherein the adhesive extends between and interconnects the back side of the fabric and the removable liner. In an embodiment, the fabric is embossed to provide an embossed fabric. In an embodiment, the fabric is embossed on the front side to provide an embossed fabric. In an embodiment, the fabric is embossed on the back side to provide an embossed fabric. In an embodiment, the fabric is embossed on the front side and on the back side to provide an embossed fabric.

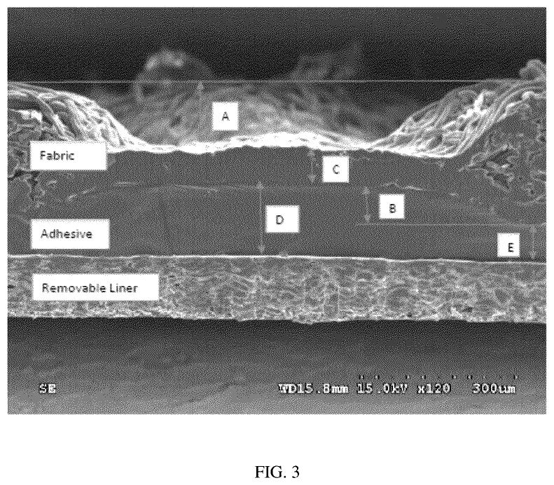

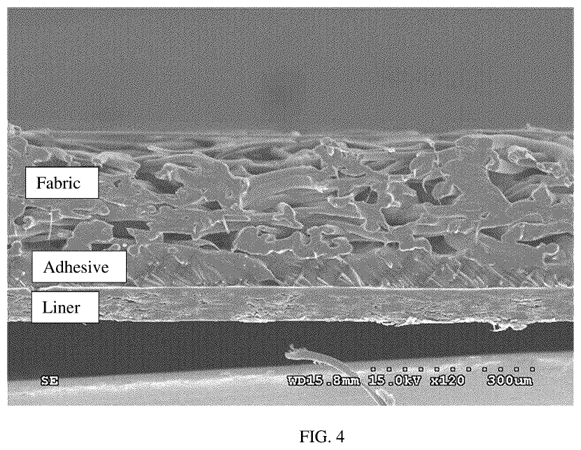

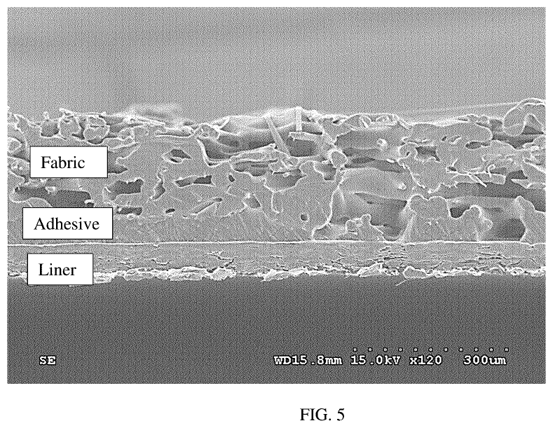

The embossed fabric includes a non-embossed portion and an embossed portion that is arranged to extend away from the non-embossed portion. On the embossed fabric, the non-embossed portion and the embossed portion provide two different depths of thickness. This concept is shown by FIG. 3 and described below. In addition, FIGS. 4 and 5 are indicative of the prior art compositions and contain a uniform thickness of adhesive.

In an embodiment, the embossed fabric can have a depth of the outer layer of the fabric that is not in contact with the adhesive. This is shown as “A” in FIG. 3 and can be referred to the “front side” of the fabric as well as the “bond depth” of the embossed portion.

In an embodiment, the embossed fabric has a depth of the inner layer of the fabric that is in contact with the adhesive. This is shown as “B” in FIG. 3 and can be referred to the “back side” of the fabric as well as the “bond depth” of the non-embossed portion.

Further, as shown as “C” in FIG. 3, a bridge can be formed by the embossing process of the fabric and can connect opposite sides of two sections of the non-embossed portions of the embossed fabric. Each of “A”, “B”, and “C” of FIG. 3 can have a particular thickness or a range of thickness as described herein.

In an embodiment, one or more repositories are present on the embossed fabric. The repositories, for instance, can be present as a result of the embossment to place various “pockets” in the embossed fabric. For instance, a repository can form on the embossed portion of the embossed fabric. For instance, a repository can form on the non-embossed portion of the embossed fabric. In an embodiment, the repositories of the embossed fabric are of variable thicknesses. As used herein, a “variable” thickness can refer to a non-random design, for instance variability that follows a pattern on a fabric. For instance, variability can refer to the pattern as a result of embossment of the embossed fabric.

In an embodiment, the adhesive is present in the one or more repositories of the embossed fabric. The adhesive can be present in the one or more repositories in the embossed fabric that are formed as a result of the embossment. In an embodiment, as shown as “D” in FIG. 3, the adhesive is present in a repository formed in the embossed portion of the embossed fabric, which is represented by the adhesive being thicker at this portion of the adhesive tape composition. Further, as shown as “E” in FIG. 3, the adhesive is present in a repository formed in the non-embossed portion of the embossed fabric, which is represented by the adhesive being thinner at this portion of the adhesive tape composition. Without being bound by any theory, it is believed that the presence of adhesive at the thicker and thinner amounts along the embossed fabric can result in the improved performance of the adhesive tape compositions of the present disclosure.

Various adhesives are contemplated for the adhesive tape compositions of the present disclosure. For instance, an adhesive can comprise, consist essentially of, or consist of a solvent-based acrylic adhesive, an acrylic-rubber hybrid adhesive, a rubber based adhesive, a polyurethane adhesive, or a silicon adhesive. Further, an adhesive can comprise, consist essentially of, or consist of a solids-based acrylic adhesive, an acrylic-rubber hybrid adhesive, a rubber based adhesive, a polyurethane adhesive, or a silicon adhesive, up to 100% solids. In one aspect, an adhesive can be indicated for long term skin wear. In an embodiment, the adhesive is an acrylic. In an embodiment, the adhesive is of medical grade.

For instance, an adhesive such as Aroset S488 can be obtained from Bostik, Inc. (Wauwatosa, Wisconsin, U.S.A.). For instance, an adhesive such as Durotack DT AH 115 can be obtained from Henkel (Düsseldorf, Germany). For instance, an adhesive such as BTX-110 can be obtained from Toyo Ink (Tokyo, Japan).

The adhesive of the adhesive tape composition can have various properties of certain ranges, including any of the following:

| Property | Range/Name | |
|---|---|---|
| Non-volatiles | 25-75% | |
| | 30-70% | |
| | 35-65% | |
| | 40-60% | |
| | 45-55% | |
| Viscosity | 100-10,000 | cP |
| | 1000-8000 | cP |
| | 1500-7000 | cP |
| | 2000-5000 | cP |
| | 2500-7000 | cP |
| | 3000-6000 | cP |
| Solvents | Ethyl acetate, toluene, | |
| | isopropanol, heptane | |
| Density | 7.0-8.0 | lbs/gal |
| Flash point range (SETA) | 20-73° | F. |

In an embodiment, the adhesive tape composition has a thickness between 8 to 20 mils (without including the removable liner) as measured by PSTC 133 Test Methods. In other embodiments, the adhesive tape composition can have a thickness, without including the removable liner, of greater than or equal to 8 mils, of greater than or equal to 8.2 mils, of greater than or equal to 8.4 mils, of greater than or equal to 8.6 mils, of greater than or equal to 8.8 mils, of greater than or equal to 9 mils, of greater than or equal to 9.2 mils, of greater than or equal to 9.4 mils, of greater than or equal to 9.6 mils, of greater than or equal to 9.8 mils, of greater than or equal to 10 mils, of greater than or equal to 10.2 mils, of greater than or equal to 10.4 mils, of greater than or equal to 10.6 mils, of greater than or equal to 10.8 mils, of greater than or equal to 11 mils, of greater than or equal to 11.2 mils, of greater than or equal to 11.4 mils, of greater than or equal to 11.6 mils, of greater than or equal to 11.8 mils, of greater than or equal to 12 mils, of greater than or equal to 12.2 mils, of greater than or equal to 12.4 mils, of greater than or equal to 12.6 mils, of greater than or equal to 12.8 mils, of greater than or equal to 13 mils, of greater than or equal to 13.2 mils, of greater than or equal to 13.4 mils, of greater than or equal to 13.6 mils, of greater than or equal to 13.8 mils, of greater than or equal to 14 mils, of greater than or equal to 14.2 mils, of greater than or equal to 14.4 mils, of greater than or equal to 14.6 mils, of greater than or equal to 14.8 mils, of greater than or equal to 15 mils, of greater than or equal to 15.2 mils, of greater than or equal to 15.4 mils, of greater than or equal to 15.6 mils, of greater than or equal to 15.8 mils, of greater than or equal to 16 mils, of greater than or equal to 16.2 mils, of greater than or equal to 16.4 mils, of greater than or equal to 16.6 mils, of greater than or equal to 16.8 mils, of greater than or equal to 17 mils, of greater than or equal to 17.2 mils, of greater than or equal to 17.4 mils, of greater than or equal to 17.6 mils, of greater than or equal to 17.8 mils, of greater than or equal to 18 mils, of greater than or equal to 18.2 mils, of greater than or equal to 18.4 mils, of greater than or equal to 18.6 mils, of greater than or equal to 18.8 mils, of greater than or equal to 19 mils, of greater than or equal to 19.2 mils, of greater than or equal to 19.4 mils, of greater than or equal to 19.6 mils, of greater than or equal to 19.8 mils, or of greater than or equal to 20 mils, including any and all ranges therebetween two stated thicknesses.

In some embodiments, the fabric can have a thickness (as measured by PSTC 133 Test Methods) of greater than or equal to 3 mils, of greater than or equal to 3.2 mils, of greater than or equal to 3.4 mils, of greater than or equal to 3.6 mils, of greater than or equal to 3.8 mils, of greater than or equal to 4 mils, of greater than or equal to 4.2 mils, of greater than or equal to 4.4 mils, of greater than or equal to 4.6 mils, of greater than or equal to 4.8 mils, of greater than or equal to 5 mils, of greater than or equal to 5.2 mils, of greater than or equal to 5.4 mils, of greater than or equal to 5.6 mils, of greater than or equal to 5.8 mils, of greater than or equal to 6 mils, of greater than or equal to 6.1 mils, of greater than or equal to 6.2 mils, of greater than or equal to 6.3 mils, of greater than or equal to 6.4 mils, of greater than or equal to 6.5 mils, of greater than or equal to 6.6 mils, of greater than or equal to 6.7 mils, of greater than or equal to 6.8 mils, of greater than or equal to 6.9 mils, of greater than or equal to 7 mils, of greater than or equal to 7.1 mils, of greater than or equal to 7.2 mils, of greater than or equal to 7.3 mils, of greater than or equal to 7.4 mils, of greater than or equal to 7.5 mils, of greater than or equal to 7.6 mils, of greater than or equal to 7.7 mils, of greater than or equal to 7.8 mils, of greater than or equal to 7.9 mils, of greater than or equal to 8 mils, of greater than or equal to 8.1 mils, of greater than or equal to 8.2 mils, of greater than or equal to 8.3 mils, of greater than or equal to 8.4 mils, of greater than or equal to 8.5 mils, of greater than or equal to 8.6 mils, of greater than or equal to 8.7 mils, of greater than or equal to 8.8 mils, of greater than or equal to 8.9 mils, of greater than or equal to 9 mils, of greater than or equal to 9.2 mils, of greater than or equal to 9.4 mils, of greater than or equal to 9.6 mils, of greater than or equal to 9.8 mils, of greater than or equal to 10 mils, of greater than or equal to 10.2 mils, of greater than or equal to 10.4 mils, of greater than or equal to 10.6 mils, of greater than or equal to 10.8 mils, of greater than or equal to 11 mils, of greater than or equal to 11.2 mils, of greater than or equal to 11.4 mils, of greater than or equal to 11.6 mils, of greater than or equal to 11.8 mils, or of greater than or equal to 12 mils, including any and all ranges therebetween two stated thicknesses. The same ranges apply to the embossed fabric, the embossed portion of the embossed fabric, and the non-embossed portion of the embossed fabric as described herein.

In some embodiments, the adhesive can have a maximum thickness (as measured by PSTC 133 Test Methods) of greater than or equal to 1 mils, of greater than or equal to 1.2 mils, of greater than or equal to 1.4 mils, of greater than or equal to 1.6 mils, of greater than or equal to 1.8 mils, of greater than or equal to 2 mils, of greater than or equal to 2.2 mils, of greater than or equal to 2.4 mils, of greater than or equal to 2.6 mils, of greater than or equal to 2.8 mils, of greater than or equal to 3 mils, of greater than or equal to 3.1 mils, of greater than or equal to 3.2 mils, of greater than or equal to 3.3 mils, of greater than or equal to 3.4 mils, of greater than or equal to 3.5 mils, of greater than or equal to 3.6 mils, of greater than or equal to 3.7 mils, of greater than or equal to 3.8 mils, of greater than or equal to 3.9 mils, of greater than or equal to 4 mils, of greater than or equal to 4.1 mils, of greater than or equal to 4.2 mils, of greater than or equal to 4.3 mils, of greater than or equal to 4.4 mils, of greater than or equal to 4.5 mils, of greater than or equal to 4.6 mils, of greater than or equal to 4.7 mils, of greater than or equal to 4.8 mils, of greater than or equal to 4.9 mils, of greater than or equal to 5 mils, of greater than or equal to 5.1 mils, of greater than or equal to 5.2 mils, of greater than or equal to 5.3 mils, of greater than or equal to 5.4 mils, of greater than or equal to 5.5 mils, of greater than or equal to 5.6 mils, of greater than or equal to 5.7 mils, of greater than or equal to 5.8 mils, of greater than or equal to 5.9 mils, of greater than or equal to 6 mils, of greater than or equal to 6.1 mils, of greater than or equal to 6.2 mils, of greater than or equal to 6.3 mils, of greater than or equal to 6.4 mils, of greater than or equal to 6.5 mils, of greater than or equal to 6.6 mils, of greater than or equal to 6.7 mils, of greater than or equal to 6.8 mils, of greater than or equal to 6.9 mils, of greater than or equal to 7 mils, of greater than or equal to 7.1 mils, of greater than or equal to 7.2 mils, of greater than or equal to 7.3 mils, of greater than or equal to 7.4 mils, of greater than or equal to 7.5 mils, of greater than or equal to 7.6 mils, of greater than or equal to 7.7 mils, of greater than or equal to 7.8 mils, of greater than or equal to 7.9 mils, of greater than or equal to 8 mils, of greater than or equal to 8.1 mils, of greater than or equal to 8.2 mils, of greater than or equal to 8.3 mils, of greater than or equal to 8.4 mils, of greater than or equal to 8.5 mils, of greater than or equal to 8.6 mils, of greater than or equal to 8.7 mils, of greater than or equal to 8.8 mils, of greater than or equal to 8.9 mils, of greater than or equal to 9 mils, of greater than or equal to 9.2 mils, of greater than or equal to 9.4 mils, of greater than or equal to 9.6 mils, of greater than or equal to 9.8 mils, or of greater than or equal to 10 mils, including any and all ranges therebetween two stated thicknesses. Furthermore, the adhesive can be present at two or more different thicknesses as described above when it is present in the embossed portion and/or the non-embossed portion of an embossed fabric.

In some embodiments, the removable liner can have a thickness of greater than or equal to 1 mils, of greater than or equal to 1.2 mils, of greater than or equal to 1.4 mils, of greater than or equal to 1.6 mils, of greater than or equal to 1.8 mils, of greater than or equal to 2 mils, of greater than or equal to 2.2 mils, of greater than or equal to 2.4 mils, of greater than or equal to 2.6 mils, of greater than or equal to 2.8 mils, of greater than or equal to 3 mils, of greater than or equal to 3.1 mils, of greater than or equal to 3.2 mils, of greater than or equal to 3.3 mils, of greater than or equal to 3.4 mils, of greater than or equal to 3.5 mils, of greater than or equal to 3.6 mils, of greater than or equal to 3.7 mils, of greater than or equal to 3.8 mils, of greater than or equal to 3.9 mils, of greater than or equal to 4 mils, of greater than or equal to 4.1 mils, of greater than or equal to 4.2 mils, of greater than or equal to 4.3 mils, of greater than or equal to 4.4 mils, of greater than or equal to 4.5 mils, of greater than or equal to 4.6 mils, of greater than or equal to 4.7 mils, of greater than or equal to 4.8 mils, of greater than or equal to 4.9 mils, of greater than or equal to 5 mils, of greater than or equal to 5.1 mils, of greater than or equal to 5.2 mils, of greater than or equal to 5.3 mils, of greater than or equal to 5.4 mils, of greater than or equal to 5.5 mils, of greater than or equal to 5.6 mils, of greater than or equal to 5.7 mils, of greater than or equal to 5.8 mils, of greater than or equal to 5.9 mils, of greater than or equal to 6 mils, of greater than or equal to 6.1 mils, of greater than or equal to 6.2 mils, of greater than or equal to 6.3 mils, of greater than or equal to 6.4 mils, of greater than or equal to 6.5 mils, of greater than or equal to 6.6 mils, of greater than or equal to 6.7 mils, of greater than or equal to 6.8 mils, of greater than or equal to 6.9 mils, of greater than or equal to 7 mils, of greater than or equal to 7.1 mils, of greater than or equal to 7.2 mils, of greater than or equal to 7.3 mils, of greater than or equal to 7.4 mils, of greater than or equal to 7.5 mils, of greater than or equal to 7.6 mils, of greater than or equal to 7.7 mils, of greater than or equal to 7.8 mils, of greater than or equal to 7.9 mils, or of greater than or equal to 8 mils, including any and all ranges therebetween two stated thicknesses.

In an embodiment, the adhesive tape composition is configured for attachment to a medical device. In an embodiment, the medical device is a glucose monitoring device. In an embodiment, the medical device is a medical diagnostic device. In an embodiment, the medical device is a smart wearable device. For these embodiments, it is contemplated that the medical device utilizes adhesive tape for attachment of the device to skin. Without being bound by any theory, the medical device can be attached to skin via adhesive tape for a prolonged time period without incurring edge lifting or adhesive oozing around the perimeter of the adhesive tape.

Furthermore, in some embodiments, the adhesive tape can be evaluated prior to use in order to analyze its biomedical or biocompatibility. For instance, the adhesive tape can under testing such as cytotoxicity, irritation, and sensitization prior to use according to the present disclosure.

Various removable liners are contemplated for the adhesive tape compositions of the present disclosure. For instance, a removable liner such as Grade 52564 80 #(lb/ream basis weight) super-calendered kraft (SCK) white paper liner can be obtained from Loparex (Cary, North Carolina, U.S.A.). Other removable liners can be obtained, for example, from MPI, Ahlstrom, MTI, and Mondi.

For instance, a removable liner can have a basis weight of between 42 to 96 lb/ream, preferably 60 to 80 lb/ream. In an embodiment, the removable liner is one side siliconized. In an embodiment, the removable liner is double side siliconized. In an embodiment, the removable liner is a poly-coated liner.

In an illustrative aspect, a medical tape composition is provided. The medical tape composition comprises i) a fabric and ii) means for coupling a medical device to an occupant. In an embodiment, the means for coupling comprises an adhesive.

In an embodiment, the means for coupling is adapted for placement on the occupant for at least 1 day without detaching. In an embodiment, the means for coupling is adapted for placement on the occupant for at least 2 days without detaching. In an embodiment, the means for coupling is adapted for placement on the occupant for at least 3 days without detaching. In an embodiment, the means for coupling is adapted for placement on the occupant for at least 5 days without detaching. In an embodiment, the means for coupling is adapted for placement on the occupant for at least 7 days without detaching. In an embodiment, the means for coupling is adapted for placement on the occupant for at least 10 days without detaching. In an embodiment, the means for coupling is adapted for placement on the occupant for at least 14 days without detaching.

In an embodiment, the medical device is a glucose monitoring device.

In an embodiment, the fabric is a non-woven fabric. In an embodiment, the fabric is a breathable fabric. In an embodiment, the fabric comprises polyurethane. In an embodiment, the fabric is configured for bi-directional stretching.

In an embodiment, the fabric comprises a front side and a back side, wherein, the adhesive extends between and interconnects the back side of the fabric and the removable liner. In an embodiment, the fabric is embossed on the front side to provide an embossed fabric. In an embodiment, the fabric is embossed on the back side to provide an embossed fabric. In an embodiment, the fabric is embossed on the front side and on the back side to provide an embossed fabric.

In an embodiment, one or more repositories are present on the embossed fabric. In an embodiment, the repositories of the embossed fabric are of variable thicknesses. In an embodiment, the adhesive is present in the one or more repositories of the embossed fabric.

The following numbered embodiments are contemplated and are non-limiting:
1. An adhesive tape composition comprising i) a fabric and ii) an adhesive.
2. The adhesive tape composition of clause 1, any other suitable clause, or any combination of suitable clauses, wherein the fabric is a non-woven fabric.
3. The adhesive tape composition of clause 1, any other suitable clause, or any combination of suitable clauses, wherein the fabric is a breathable fabric.
4. The adhesive tape composition of clause 1, any other suitable clause, or any combination of suitable clauses, wherein the fabric comprises polyurethane or a polyurethane resin.
5. The adhesive tape composition of clause 1, any other suitable clause, or any combination of suitable clauses, wherein the adhesive tape composition further comprises a removable liner.
6. The adhesive tape composition of clause 1, any other suitable clause, or any combination of suitable clauses, wherein the fabric comprises polyurethane resin compounded with one or more additives.
7. The adhesive tape composition of clause 6, any other suitable clause, or any combination of suitable clauses, wherein the additive is selected from the group consisting of a colorant, a dye, a pigment, an ultraviolet (UV) blocker, a UV absorber, or a functional filler, a polymer, and any combination thereof.
8. The adhesive tape composition of clause 1, any other suitable clause, or any combination of suitable clauses, wherein the fabric is configured for bi-directional stretching.
9. The adhesive tape composition of clause 1, any other suitable clause, or any combination of suitable clauses, wherein the fabric comprises a front side and a back side, wherein the adhesive extends between and interconnects the back side of the fabric and the removable liner.
10. The adhesive tape composition of clause 9, any other suitable clause, or any combination of suitable clauses, wherein the fabric is embossed on the front side to provide an embossed fabric.
11. The adhesive tape composition of clause 9, any other suitable clause, or any combination of suitable clauses, wherein the fabric is embossed on the back side to provide an embossed fabric.
12. The adhesive tape composition of clause 9, any other suitable clause, or any combination of suitable clauses, wherein the fabric is embossed on the front side and on the back side to provide an embossed fabric.
13. The adhesive tape composition of clause 9, any other suitable clause, or any combination of suitable clauses, wherein the fabric is an embossed fabric.
14. The adhesive tape composition of clause 13, any other suitable clause, or any combination of suitable clauses, wherein the embossed fabric comprises a non-embossed portion and an embossed portion.
15. The adhesive tape composition of clause 13, any other suitable clause, or any combination of suitable clauses, wherein one or more repositories are present on the embossed fabric.
16. The adhesive tape composition of clause 15, any other suitable clause, or any combination of suitable clauses, wherein the repositories of the embossed fabric are of variable thicknesses.
17. The adhesive tape composition of clause 15, any other suitable clause, or any combination of suitable clauses, wherein the adhesive is present in the one or more repositories of the embossed fabric.
18. The adhesive tape composition of clause 1, any other suitable clause, or any combination of suitable clauses, wherein the adhesive comprises a solvent-based acrylic adhesive.
19. The adhesive tape composition of clause 1, any other suitable clause, or any combination of suitable clauses, wherein the adhesive comprises a solids-based acrylic adhesive.
20. The adhesive tape composition of clause 1, any other suitable clause, or any combination of suitable clauses, wherein the adhesive comprises an acrylic-rubber hybrid adhesive.
21. The adhesive tape composition of clause 1, any other suitable clause, or any combination of suitable clauses, wherein the adhesive comprises a rubber based adhesive
22. The adhesive tape composition of clause 1, any other suitable clause, or any combination of suitable clauses, wherein the adhesive comprises a polyurethane adhesive.
23. The adhesive tape composition of clause 1, any other suitable clause, or any combination of suitable clauses, wherein the adhesive comprises a silicon adhesive.
24. The adhesive tape composition of clause 1, any other suitable clause, or any combination of suitable clauses, wherein the adhesive is indicated for long term skin wear.
25. The adhesive tape composition of clause 1, any other suitable clause, or any combination of suitable clauses, wherein the adhesive is an acrylic.
26. The adhesive tape composition of clause 1, any other suitable clause, or any combination of suitable clauses, wherein the adhesive is of medical grade.
27. The adhesive tape composition of clause 1, any other suitable clause, or any combination of suitable clauses, wherein the adhesive tape composition is configured for attachment to a medical device.
28. The adhesive tape composition of clause 27, any other suitable clause, or any combination of suitable clauses, wherein the medical device is a glucose monitoring device.
29 The adhesive tape composition of clause 27, any other suitable clause, or any combination of suitable clauses, wherein the medical device is a medical diagnostic device.
30. The adhesive tape composition of clause 27, any other suitable clause, or any combination of suitable clauses, wherein the medical device is a smart wearable device.
31. An adhesive tape composition comprising i) a fabric and ii) means for coupling a medical device to an occupant.
32. The adhesive tape composition of clause 31, any other suitable clause, or any combination of suitable clauses, wherein the means for coupling comprises an adhesive.
33. The adhesive tape composition of clause 32, any other suitable clause, or any combination of suitable clauses, wherein the adhesive comprises a solvent-based acrylic adhesive.
34. The adhesive tape composition of clause 32, any other suitable clause, or any combination of suitable clauses, wherein the adhesive comprises a solids-based acrylic adhesive.
35. The adhesive tape composition of clause 32, any other suitable clause, or any combination of suitable clauses, wherein the adhesive comprises an acrylic-rubber hybrid adhesive.
36. The adhesive tape composition of clause 32, any other suitable clause, or any combination of suitable clauses, wherein the adhesive comprises a rubber based adhesive.
37. The adhesive tape composition of clause 32, any other suitable clause, or any combination of suitable clauses, wherein the adhesive comprises a polyurethane adhesive.
38. The adhesive tape composition of clause 32, any other suitable clause, or any combination of suitable clauses, wherein the adhesive comprises a silicon adhesive.
39. The adhesive tape composition of clause 32, any other suitable clause, or any combination of suitable clauses, wherein the adhesive is indicated for long term skin wear.
40 The adhesive tape composition of clause 32, any other suitable clause, or any combination of suitable clauses, wherein the adhesive is an acrylic.
41. The adhesive tape composition of clause 32, any other suitable clause, or any combination of suitable clauses, wherein the adhesive is of medical grade.
42. The adhesive tape composition of clause 31, any other suitable clause, or any combination of suitable clauses, wherein the adhesive tape composition is configured for attachment to a medical device.
43. The adhesive tape composition of clause 42, any other suitable clause, or any combination of suitable clauses, wherein the medical device is a glucose monitoring device.
44. The adhesive tape composition of clause 42, any other suitable clause, or any combination of suitable clauses, wherein the medical device is a medical diagnostic device.
45. The adhesive tape composition of clause 42, any other suitable clause, or any combination of suitable clauses, wherein the medical device is a smart wearable device.
46. The adhesive tape composition of clause 31, any other suitable clause, or any combination of suitable clauses, wherein the means for coupling is adapted for placement on the occupant for at least 1 day without detaching.
47. The adhesive tape composition of clause 31, any other suitable clause, or any combination of suitable clauses, wherein the means for coupling is adapted for placement on the occupant for at least 2 days without detaching.
48. The adhesive tape composition of clause 31, any other suitable clause, or any combination of suitable clauses, wherein the means for coupling is adapted for placement on the occupant for at least 3 days without detaching.
49. The adhesive tape composition of clause 31, any other suitable clause, or any combination of suitable clauses, wherein the means for coupling is adapted for placement on the occupant for at least 5 days without detaching.
50. The adhesive tape composition of clause 31, any other suitable clause, or any combination of suitable clauses, wherein the means for coupling is adapted for placement on the occupant for at least 7 days without detaching.
51. The adhesive tape composition of clause 31, any other suitable clause, or any combination of suitable clauses, wherein the means for coupling is adapted for placement on the occupant for at least 10 days without detaching.
52. The adhesive tape composition of clause 31, any other suitable clause, or any combination of suitable clauses, wherein the means for coupling is adapted for placement on the occupant for at least 14 days without detaching.
53. The adhesive tape composition of clause 31, any other suitable clause, or any combination of suitable clauses, wherein the medical device is a glucose monitoring device.
54. The adhesive tape composition of clause 31, any other suitable clause, or any combination of suitable clauses, wherein the fabric is a non-woven fabric.
55. The adhesive tape composition of clause 31, any other suitable clause, or any combination of suitable clauses, wherein the fabric is a breathable fabric.
56. The adhesive tape composition of clause 31, any other suitable clause, or any combination of suitable clauses, wherein the fabric comprises polyurethane.
57. The adhesive tape composition of clause 31, any other suitable clause, or any combination of suitable clauses, wherein the fabric comprises polyurethane resin.
58. The adhesive tape composition of clause 31, any other suitable clause, or any combination of suitable clauses, wherein the fabric comprises polyurethane resin compounded with one or more additives.
59. The adhesive tape composition of clause 58, any other suitable clause, or any combination of suitable clauses, wherein the additive is selected from the group consisting of a colorant, a dye, a pigment, an ultraviolet (UV) blocker, a UV absorber, or a functional filler, a polymer, and any combination thereof.
60. The adhesive tape composition of clause 31, any other suitable clause, or any combination of suitable clauses, wherein the fabric is configured for bi-directional stretching.
61. The adhesive tape composition of clause 31, any other suitable clause, or any combination of suitable clauses, wherein the fabric comprises a front side and a back side, wherein the adhesive extends between and interconnects the back side of the fabric and the removable liner.
62. The adhesive tape composition of clause 61, any other suitable clause, or any combination of suitable clauses, wherein the fabric is embossed on the front side to provide an embossed fabric.
63. The adhesive tape composition of clause 61, any other suitable clause, or any combination of suitable clauses, wherein the fabric is embossed on the back side to provide an embossed fabric.
64. The adhesive tape composition of clause 61, any other suitable clause, or any combination of suitable clauses, wherein the fabric is embossed on the front side and on the back side to provide an embossed fabric.
65. The adhesive tape composition of clause 61, any other suitable clause, or any combination of suitable clauses, wherein the fabric is an embossed fabric.
66. The adhesive tape composition of clause 65, any other suitable clause, or any combination of suitable clauses, wherein the embossed fabric comprises a non-embossed portion and an embossed portion.
67. The adhesive tape composition of clause 65, any other suitable clause, or any combination of suitable clauses, wherein one or more repositories are present on the embossed fabric.
68 The adhesive tape composition of clause 67, any other suitable clause, or any combination of suitable clauses, wherein the repositories of the embossed fabric are of variable thicknesses.
69 The adhesive tape composition of clause 67, any other suitable clause, or any combination of suitable clauses, wherein the adhesive is present in the one or more repositories of the embossed fabric.
70. An adhesive tape composition comprising i) an embossed fabric, ii) an adhesive in contact with a side of the embossed fabric, wherein the embossed fabric comprises variable adhesive thickness.
71. The adhesive tape composition of clause 70, any other suitable clause, or any combination of suitable clauses, wherein the adhesive tape composition further comprises a removable liner.

### EXAMPLES

### Example 1

### Formation and Analysis of Adhesive Tape Compositions

An exemplary adhesive tape composition can be formed as follows. Adhesive was uniformly coated on the siliconized side of an 80 lb basis weight SCK paper release liner using steel coating rollers at the thickness of 3.5 mil. This was air dried for 15 minutes then dried at 70° C. for 15 minutes, followed by the final drying step at 121° C. for 15 minutes. Dried adhesive on the liner was then laminated on the adhesive side of the 75 gsm, wherein both sides were embossed polyurethane fabric under laminating pressure of 20 psa.

The unique profile of the adhesive tape is formed after laminating step on the boundary of the dried adhesive and the embossed side surface of the fabric. After lamination to the fabric, the adhesive thickness varies along and across the coated web due to visco-elastic nature of the adhesive as it follows the embossing topography of the fabric.

Four prior art adhesive tape compositions that did not include embossment of fabrics were analyzed as shown in Table 1:

TABLE 1
Prior Art Adhesive Tape Compositions

| Sample Number | Sample Name | Embossment |
|---|---|---|
| 1 | 3M, 9907T | None |
| 2 | 3M, 9907W | None |
| 3 | Avery, MED 5740 | None |
| 4 | Dermamed, DM2275 | None |

Three adhesive tape compositions that included embossment of fabrics were analyzed as shown in Table 2:

TABLE 2
Embossed Adhesive Tape Compositions

| Sample Number | Sample Name | Embossment |
|---|---|---|
| 5 | 3619A BR688 | Front and back sides |
| 6 | 3620A BR1114 | Front and back sides |
| 7 | 3621A BR583 | Front side |

The various adhesive tape compositions were analyzed to evaluate bond depth (for embossed side and non-embossed side, as applicable), bond site thickness, unbonded area, surface roughness, and fiber size. The results of the analyses are shown in Table 3.

TABLE 3

| | | | Bond | | | | |
|---|---|---|---|---|---|---|---|
| | | Bond | Depth | | | | |
| | | Depth | (non- | | | Surface | |
| Sample | Sample | (embossed | embossed | Bond Site | Unbonded | Rough- | Fiber |
| Number | Name | side) | side) | Thickness | Area | ness | Size |
| 1 | 3M, | n/a | n/a | n/a | n/a | 59.5 μm | 11.4 μm |
| | 9907T | | | | | | |
| 2 | 3M, | n/a | n/a | n/a | n/a | 61.5 μm | 11.6 μm |
| | 9907W | | | | | | |
| 3 | Avery, | n/a | n/a | n/a | 76.3% | 37.3 μm | 8.3 μm |
| | MED 5740 | | | | | | |
| 4 | Dermamed, | n/a | n/a | n/a | n/a | 29.1 μm | 9.6 μm |
| | DM2275 | | | | | | |
| 5 | 3619A | 88 μm | 50 μm | 32 μm | 77.2% | 53.7 μm | 12.1 μm |
| | BR688 | | | | | | |
| 6 | 3620A | 90 μm | 47 μm | 30 μm | 76.7% | 55.2 μm | 11.4 μm |
| | BR1114 | | | | | | |
| 7 | 3621A | 75 μm | n/a | 106 μm | 73.9% | 38.1 μm | 8.1 μm |
| | BR583 | | | | | | |

Adhesive tape compositions 5 and 6 (i.e., those with embossed fabric that was embossed on the front side and the back side and demonstrating variable thickness of adhesive) demonstrated improved skin wear duration compared to the prior art samples 1-4. Adhesive tape compositions 5 and 6 also demonstrated improved support of a medical device when the medical device was attached to the skin of a user using these adhesive tape compositions.

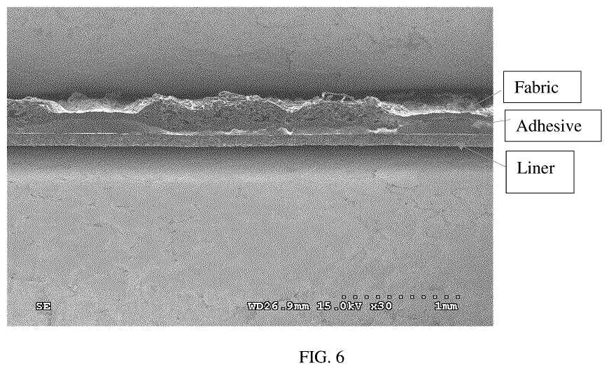

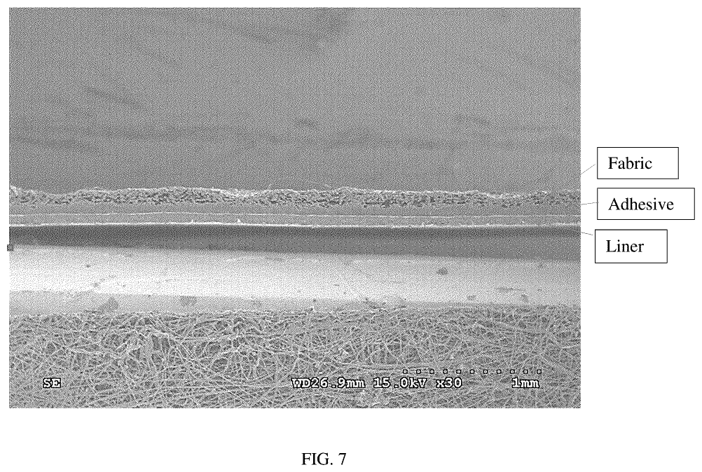

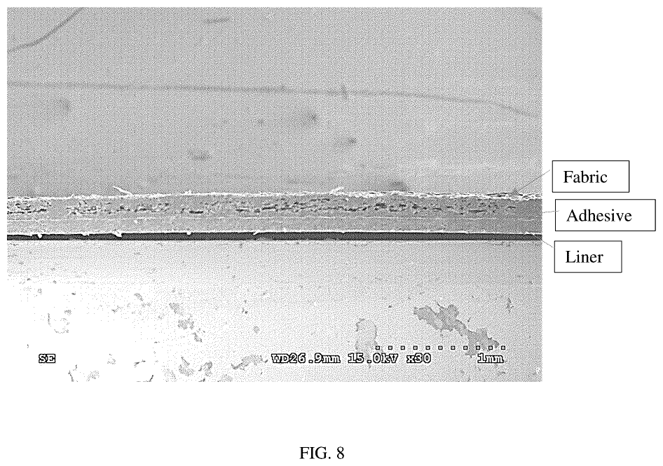

FIG. 6 shows the variable thickness of adhesive in view of the embossed pattern on Sample 5 (i.e., 3619A). In contrast, FIGS. 7 and 8 show a uniform adhesive thickness for Samples 3 and 4, respectively (i.e., Avery, MED 5740 and Dermamed, DM2275).

### Example 2

### Measurement Procedure of Adhesive Tape Compositions

The instant example provides exemplary methods for determining depth of embossed areas and other features of the fabric of adhesive tape compositions. Equipment for the instant example included Hitachi S-3000N Scanning Electron Microscope (with PCI Quartz Software), a Denton vacuum desk V coater with gold/palladium target, Max Pro Freeze Spray, aluminum regular duty single edge blades, and double slotted vise holders.

Preparation steps of the instant example included:
1. Freeze a small section of adhesive tape composition sample and then carefully cut a cross section of the sample making sure to cut through the middle of the embossed area.
2. Place the cross-sectioned sample with the cut side up in a double slotted vise holder. Repeat for additional samples.
3. Place the holders into the vacuum coater, coat the samples for 45 seconds and remove after coating is complete.

Analysis steps of the instant example included:
1. Place the coated sample in the SEM.
2. One the SEM has reached the appropriate pressure, turn on the beam, and set the sample to the following parameters:
   Working Distance=15.8 mm
   Accelerating Voltage=15 kv
   Magnification=120×
   Scale=300 μm
3. Obtain enough images to make necessary measurements. More than one area from a sample is needed to complete measurements.
4. Once all images are taken, the following measurements were made calibrated software. All measurements are made at the center of the embossed areas:
   Embossed Depth on embossed side and opposite side
   Bridge Site Thickness

### Example 3

### Measurement Analyses of Adhesive Tape Compositions

The instant example provides analyses of adhesive tape compositions according to the procedures in Example 2. Various measurements of adhesive tape compositions were undertaken for the elements shown in the SEM image of FIG. 3.

In particular, the following measurements of exemplary adhesive tape compositions of the present disclosure were made (referencing FIG. 3):

TABLE 4

| Element | Description |
|---|---|
| A | Embossment depth thickness of outer layer of fabric |
| B | Embossment depth thickness of inner layer of fabric |
| C | Bridge (Bond) site thickness |
| D | Adhesive thickness in repository formed by embossed |
| | portion of embossed fabric |
| E | Adhesive thickness in repository formed by non- |
| | embossed portion of embossed fabric |

A first exemplary adhesive tape composition (10 samples; white fabric) was analyzed under a microscope (WD 15.8 mm, 15 kV, ×120, at 300 μm scale). The thickness values at each element were measured and are shown in Table 5.

TABLE 5

| | A | B | C | D | E |
|---|---|---|---|---|---|
| Sample | (μm) | (μm) | (μm) | (μm) | (μm) |
| 1 | 65 | 41 | 25 | 149 | 69 |
| 2 | 68 | 45 | 27 | 144 | 89 |
| 3 | 117 | 78 | 35 | 179 | 91 |
| 4 | 93 | 40 | 24 | 147 | 88 |
| 5 | 91 | 50 | 37 | 223 | 117 |
| 6 | 108 | 50 | 31 | 162 | 94 |
| 7 | 114 | 48 | 29 | 207 | 84 |
| 8 | 75 | 33 | 33 | 200 | 96 |
| 9 | 78 | 52 | 35 | 177 | 89 |
| 10 | 93 | 36 | 27 | 172 | 93 |
| Average | 90 | 47 | 30 | 176 | 91 |
| Minimum | 65 | 33 | 24 | 144 | 69 |
| Maximum | 117 | 78 | 37 | 223 | 117 |

A second exemplary adhesive tape composition (10 samples; tan fabric) was analyzed under a microscope (WD 15.8 mm, 15 kV, ×120, at 300 μm scale). The thickness values at each element were measured and are shown in Table 6.

TABLE 6

| | A | B | C | D | E |
|---|---|---|---|---|---|
| Sample | (μm) | (μm) | (μm) | (μm) | (μm) |
| 1 | 68 | 66 | 30 | 190 | 124 |
| 2 | 83 | 48 | 30 | 159 | 129 |
| 3 | 122 | 61 | 28 | 192 | 93 |
| 4 | 89 | 45 | 33 | 169 | 86 |
| 5 | 81 | 43 | 36 | 159 | 68 |
| 6 | 88 | 45 | 32 | 177 | 68 |
| 7 | 91 | 50 | 28 | 162 | 84 |
| 8 | 76 | 50 | 38 | 172 | 73 |
| 9 | 81 | 38 | 27 | 154 | 111 |
| 10 | 97 | 52 | 38 | 170 | 73 |
| Average | 88 | 50 | 32 | 170 | 91 |
| Minimum | 68 | 38 | 27 | 154 | 68 |
| Maximum | 122 | 66 | 38 | 192 | 129 |

A summary of the analysis of seven exemplary adhesive tape compositions that were evaluated is found in Table 7.

TABLE 7

| | A | B | C | D | E |
|---|---|---|---|---|---|
| | (μm) | (μm) | (μm) | (μm) | (μm) |
| Preferred | 82 | 50 | 30 | 170 | 91 |
| Minimum | 46 | 23 | 21 | 144 | 68 |
| Maximum | 131 | 101 | 73 | 223 | 129 |

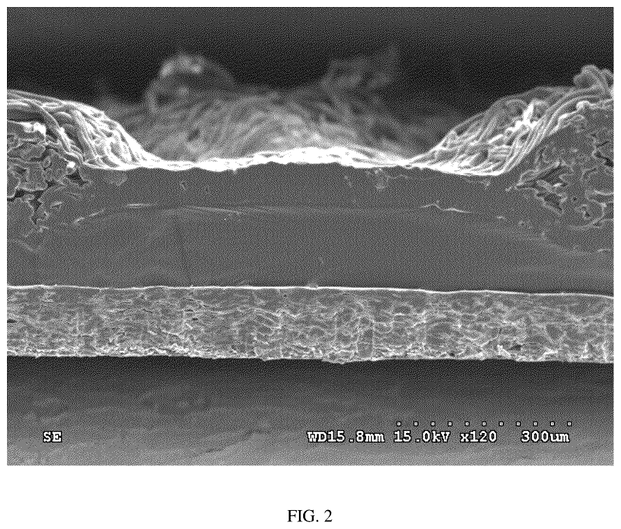

In comparison to the exemplary adhesive tape compositions shown in the SEM images of FIGS. 2 and 3, SEM images of prior art compositions 3M 9907T and 3M 9907W are shown in FIGS. 4 and 5, respectively. As shown in FIG. 4, the prior art composition 3M 9907T has a uniform thickness of fabric and adhesive. As shown in FIG. 5, the prior art composition 3M 9907W also has a uniform thickness of fabric and adhesive. These prior art compositions do not provide the improvements observed with the adhesive tape compositions of the present disclosure.

### Example 4

### Measurement of Embossment Depth of Exemplary Fabrics

The instant example provides analyses of embossment depths of fabrics. An exemplary embossed non-woven fabric sample can be evaluated. Equipment utilized for the instant example included a Hitachi S-3000N scanning electron microscope with PCI quartz software, a Denton vacuum desk V coater with gold/palladium target, a max pro freeze spray, aluminum regular duty single edge blades, and double slotted vise holders.

The fabric sample is taken from the center of the web for fabric rolls at the beginning, middle, and end of the production run. A small section of the sample is frozen by spraying the max pro freeze spray directly on the section of the fabric to be analyzed. The sample is ready for further use immediately after spray freezing.

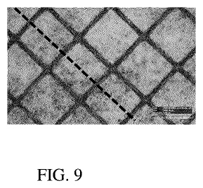

Thereafter, a cross section of the sample is cut with a razor, making sure to cut through the middle point of the embossed area. A line of the cut to be made is shown by the dashed line in FIG. 9.

The cross-sectioned sample is placed with the cut side up in a double slotted vise holder and repeated for additional samples. The holders are placed into the vacuum coater. The samples are coated for 45 seconds and then removed after coating is complete.

For testing, the coated sample are placed in the SEM chamber. Once the SEM chamber has reached the appropriate pressure, the beam is turned on and the sample is set to the following parameters:
Working Distance=15.8 mm
Accelerating Voltage=15 kv
SEM Magnification=×120
Scale=300 μm

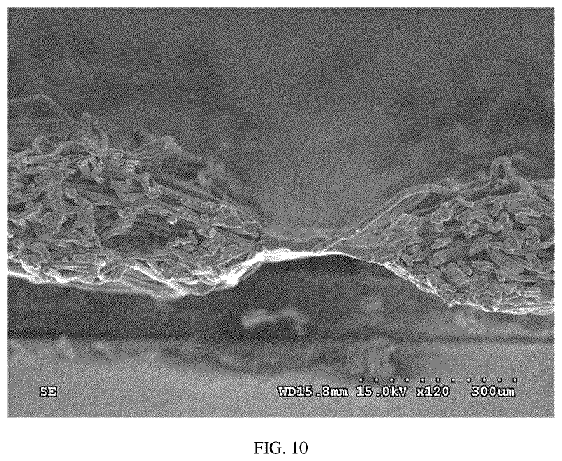

About 10 images per sample are obtained to make necessary measurements, often obtaining more than one area from a sample to complete measurements. An example of an original image is shown in FIG. 10.

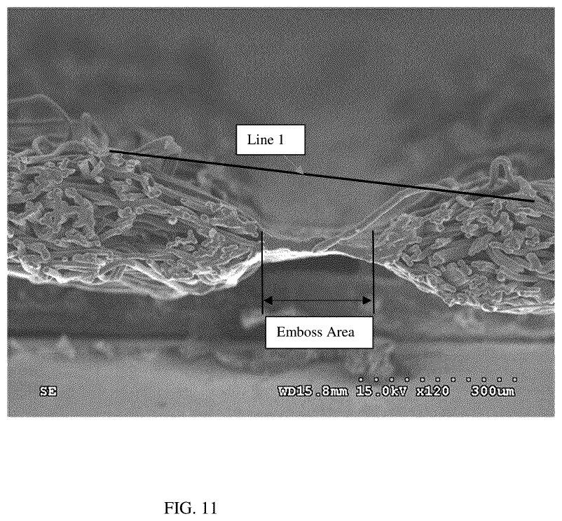

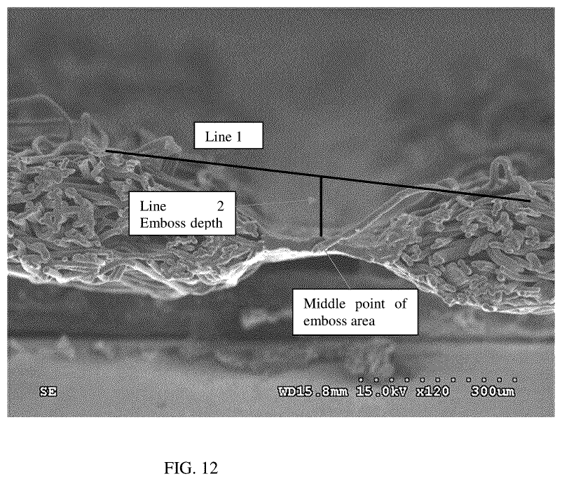

After all images are taken, the emboss depth is measured using calibrated software. All measurements are made at the center of the emboss area using following steps.
1. Referring to FIG. 11, draw Line 1 between the two highest points of fabric surface on both sides of the emboss area. Disregard individual stray fibers and loops and identify location of emboss area.
2. Referring to FIG. 12, visually identify the middle point of the emboss area. Start drawing Line 2 up from the middle point of the emboss area. Line 2 does not have to be perpendicular to Line 1, but has to start from the middle point of emboss area. The length of Line 2 is the Emboss Depth (measured in microns).
3. Record individual measurements of emboss depth and report an average of 30 measurements.

1. An adhesive tape composition comprising i) a fabric and ii) an adhesive.

2. The adhesive tape composition of claim 1, wherein the fabric is a non-woven fabric.

3. The adhesive tape composition of claim 1, wherein the adhesive tape composition further comprises a removable liner.

4. The adhesive tape composition of claim 1, wherein the fabric comprises polyurethane or polyurethane resin.

5. The adhesive tape composition of claim 1, wherein the fabric comprises polyurethane resin compounded with one or more additives.

6. The adhesive tape composition of claim 5, wherein the additive is selected from the group consisting of a colorant, a dye, a pigment, an ultraviolet (UV) blocker, a UV absorber, or a functional filler, a polymer, and any combination thereof.

7. The adhesive tape composition of claim 1, wherein the fabric comprises a front side and a back side, wherein the adhesive extends between and interconnects the back side of the fabric and the removable liner.

8. The adhesive tape composition of claim 1, wherein the fabric is an embossed fabric, wherein the embossed fabric comprises a non-embossed portion and an embossed portion.

9. The adhesive tape composition of claim 8, wherein one or more repositories are present on the embossed fabric.

10. The adhesive tape composition of claim 9, wherein the repositories of the embossed fabric are of variable thicknesses.

11. The adhesive tape composition of claim 9, wherein the adhesive is present in the one or more repositories of the embossed fabric.

12. An adhesive tape composition comprising i) a fabric and ii) means for coupling a medical device to an occupant.

13. The adhesive tape composition of claim 12, wherein the means for coupling comprises an adhesive.

14. The adhesive tape composition of claim 12, wherein the means for coupling is adapted for placement on the occupant for at least 7 days without detaching.

15. The adhesive tape composition of claim 12, wherein the means for coupling is adapted for placement on the occupant for at least 14 days without detaching.

16. The adhesive tape composition of claim 12, wherein the fabric comprises a front side and a back side, wherein the adhesive extends between and interconnects the back side of the fabric and the removable liner.

17. The adhesive tape composition of claim 16, wherein the fabric is embossed on the front side and on the back side to provide an embossed fabric, wherein the embossed fabric comprises a non-embossed portion and an embossed portion.

18. The adhesive tape composition of claim 17, wherein one or more repositories are present on the embossed fabric.

19. The adhesive tape composition of claim 18, wherein the repositories of the embossed fabric are of variable thicknesses.

20. The adhesive tape composition of claim 18, wherein the adhesive is present in the one or more repositories of the embossed fabric.
